(19) United States
(12) Patent Application Publication
(10) Pub. No.: US 2026/0124123 A1
(43) Pub. Date: May 7, 2026

(54) STABLE OIL-IN-WATER MICROEMULSION

(71) Applicant: DERMOSCIENCES FRANCE, Cagnes-Sur-Mer (FR)

(72) Inventors: Monory POT, Cagnes sur Mer (FR); Marie-Anne ACHILLE, Cagnes sur Mer (FR)

(21) Appl. No.: 19/380,792

(22) Filed: Nov. 5, 2025

(30) Foreign Application Priority Data
Nov. 6, 2024 (FR) 2412132

(51) Int. Cl. A61K 8/06; A61K 8/34; A61K 8/37; A61K 8/49; A61Q 19/00

(52) U.S. Cl. CPC A61K 8/068; A61K 8/345; A61K 8/375; A61K 8/4993; A61Q 19/00

(57) ABSTRACT

The present invention relates to a composition in the form of an oil-in-water microemulsion with excellent stability over time. In particular, the composition according to the invention comprises a medium-chain fatty acid triglyceride, a low HLB surfactant, a high HLB surfactant, a diol chosen from 1,3-propanediol and propylene glycol, and water. The invention also relates to the cosmetic use of said composition and a method for applying said composition to keratinous materials.

### TECHNICAL FIELD

The present invention relates to a composition in the form of a oil-in-water microemulsion with excellent stability over time. In particular, the composition according to the invention comprises a medium-chain fatty acid triglyceride, a low HLB surfactant, a high HLB surfactant, a diol, and water. The invention also relates to the cosmetic use of said composition and a method for applying said composition to keratinous materials.

### PRIOR ART

Cosmetic and dermatological preparations intended for skin and appendage care generally contain oil-soluble and water-soluble ingredients, often in the form of emulsions. An emulsion is a mixture of two or more immiscible or partially miscible liquids, where one liquid, called the dispersed phase or discontinuous phase, is dispersed in small droplets in the other, called the continuous phase. Emulsions are classified based on their continuous phase, into oil-in-water (O/W) or water-in-oil (W/O) emulsions.

Microemulsions represent a specific category of emulsions, obtained from four components: an aqueous phase, an oily phase, a surfactant, and a co-surfactant. The resulting composition leads to a microstructure formed of microdroplets with a size of less than 100 nanometers. This reduced droplet size allows light to pass through the microemulsion without being scattered, giving it a transparent appearance, unlike the opaque appearance of conventional emulsions.

This property gives microemulsions significant advantages in cosmetic formulations. They offer better penetration of actives due to the small droplet size, optical transparency due to the absence of light scattering, and the possibility of being prepared cold. This latter characteristic reduces energy consumption during manufacturing and allows the use of thermosensitive actives that could be degraded in a conventional emulsion. Another advantage of microemulsions is their ability to contain a larger amount of difficult-to-solubilize actives.

Despite these advantages, the long-term stability of microemulsions remains a major technical challenge. Although generally considered thermodynamically stable, microemulsions can be subject to destabilization phenomena during storage, such as sedimentation or coalescence. These processes can alter the physicochemical properties and effectiveness of the cosmetic product over time. While increasing the concentration of surfactant can improve the stability of microemulsions, high concentrations can result in a coarse, sticky, or greasy texture, thus altering the organoleptic properties of the product.

Shah et al describes the formation of microemulsions from medium-chain triglycerides as the lipophilic phase and polysorbates as surfactants (A. Shah et al, Drug Development and Industrial Pharmacy, 2017, vol. 44, pages 215-223). These microemulsions only form at very low lipophilic phase/surfactant ratios (1:9 or 2:8), thus requiring very high amounts of surfactants. The document also describes microemulsions prepared from a polysorbate, a medium-chain triglyceride, and a monoglyceride. By reproducing the teaching of this document, the Applicant observed that these microemulsions tend to lose their stability over time.

The document US2011/159104 describes honey-based microemulsions and apicultural products comprising triglycerides, low and high HLB surfactants, as well as co-solvents and water.

There is therefore a need to develop a composition in the form of a microemulsion, particularly for cosmetic and/or dermatological applications, which combines the advantages of microemulsions in terms of effectiveness and organoleptic properties, while offering improved stability capable of withstanding long-term storage conditions.

### SUMMARY OF THE INVENTION

According to a first aspect, the invention relates to a composition in the form of an oil-in-water microemulsion comprising at least:
a) a medium-chain fatty acid triglyceride;
b) a low HLB surfactant;
c) a high HLB surfactant;
d) a diol chosen from 1,3-propanediol and propylene glycol;
e) water.

Advantageously, the low HLB surfactant has an HLB ranging from 3 to 6, preferably from 5 to 6, and/or the high HLB surfactant has an HLB ranging from 13 to 20, preferably from 14 to 18.

Advantageously, the mass ratio of high HLB surfactant to the mixture (medium-chain fatty acid triglyceride+low HLB surfactant) is greater than or equal to 0.85, preferably ranges from 0.90 to 5, more preferably from 0.90 to 2.

According to a particular aspect, the invention relates to a composition in the form of an oil-in-water microemulsion comprising at least:
a) a medium-chain fatty acid triglyceride having 6 to 12 carbon atoms;
b) a low HLB surfactant having an HLB ranging from 3 to 6;
c) a high HLB surfactant having an HLB ranging from 13 to 20;
d) a diol chosen from 1,3-propanediol and propylene glycol;
e) water,
wherein the mass ratio of the high HLB surfactant to the mixture (medium-chain fatty acid triglyceride having 6 to 12 carbon atoms+low HLB surfactant) is greater than or equal to 0.85.

Advantageously, the composition according to the invention comprises from 3% to 20% by weight, preferably from 3% to 15% by weight, more preferably from 4% to 12% by weight, even more preferably from 5% to 10% by weight of diol relative to the total weight of the composition.

According to one embodiment, the diol is 1,3-propanediol.

Advantageously, the medium-chain fatty acid triglyceride is caprylic/capric triglyceride.

Advantageously, the low HLB surfactant comprises a monoglyceride and/or a diglyceride of medium-chain fatty acid, preferably chosen from glyceryl monocaprate, glyceryl dicaprate, glyceryl monocaprylate, glyceryl dicaprylate, glyceryl caprate, glyceryl caprylate, glyceryl monocaprylate/caprate, glyceryl caprylate/caprate, glyceryl dicaprylate/caprate, more preferably is glyceryl caprylate.

According to one embodiment, the high HLB surfactant comprises an ethoxylated sorbitan ester, preferably polysorbate 80.

Advantageously, the composition according to the invention comprises, relative to the total weight of the composition:
from 7% to 9% by weight of medium-chain fatty acid triglyceride;
from 12% to 15% by weight of low HLB surfactant;
from 20% to 24% by weight of high HLB surfactant;
from 5% to 10% by weight of diol; and
from 42% to 56% by weight of water.

Advantageously, the composition according to the invention further comprises at least one cosmetically and/or dermatologically acceptable agent and/or at least one biological active.

The invention also relates to the use of a composition according to the invention as a makeup, hygiene, cleaning, and/or care product for keratinous materials, preferably the skin.

The invention also relates to the non-therapeutic cosmetic use of a composition according to the invention to prevent and/or treat signs of skin aging, preferably to reduce and/or eliminate and/or attenuate wrinkles and fine lines.

The invention also relates to the non-therapeutic cosmetic use of a composition according to the invention to prevent and/or reduce and/or eliminate acne and/or to promote the elimination of cosmetic effects of acne or acne spots.

The invention also relates to the non-therapeutic cosmetic use of a composition according to the invention to prevent and/or reduce and/or attenuate and/or eliminate pigmentation spots, and/or reduce their size, and/or unify and/or illuminate and/or brighten the complexion.

The Applicant has developed stable oil-in-water microemulsion compositions by optimizing the choice of ingredients and their proportions. The microemulsions according to the invention have shown exceptional stability over several months, even years, even when subjected to high storage temperatures.

### DETAILED DESCRIPTION

The expression “consists essentially of” followed by one or more features means that, in addition to the explicitly listed components or steps, components or steps that do not significantly alter the properties and characteristics of the invention may be included in the process or material of the invention.

The expression “comprised between X and Y” includes the bounds, unless explicitly stated otherwise. This expression therefore means that the intended range comprises the values X, Y, and all values from X to Y.

The different embodiments, variants, preferences, and advantages described for each of the objects of the invention apply to all objects of the invention and can be taken separately or in combination.

In the sense of the present invention, “microemulsion” means a transparent or translucent colloidal system consisting of two immiscible phases, notably a lipophilic phase and a hydrophilic phase, stabilized by a surfactant or a mixture of a surfactant and a co-surfactant. Unlike conventional emulsions, which can be opalescent or turbid, microemulsions are clear and homogeneous with a diameter of the dispersed domains or droplets ranging from 1 to 100 nm.

In the following description and claims, the term “microemulsion” exclusively refers to an oil-in-water emulsion. This means that the droplets of the lipophilic phase are dispersed in the aqueous hydrophilic phase, which constitutes the continuous phase.

In the sense of the invention “lipophilic” means a phase and/or a compound that has an affinity or a tendency to mix with fats, oils, and/or non-polar solvents.

In the sense of the invention “hydrophilic” means a phase and/or a compound that has an affinity or a tendency to mix with water or aqueous solutions. In the context of the invention, the aqueous hydrophilic phase is the continuous phase of the microemulsion.

In the sense of the invention, “surfactant” or “emulsifier mean a compound possessing an amphiphilic structure capable of reducing the surface tension between two immiscible phases, notably a lipophilic phase and a hydrophilic phase, thus allowing the formation and stabilization of droplets of one of the phases dispersed in the other.

The term “HLB,” also called “Hydrophilic-Lipophilic Balance”, refers to the value that characterizes the balance between the hydrophilic part and the lipophilic part of a surfactant molecule. The HLB value is generally determined by calculating the percentages of the molecular weights of the hydrophilic and lipophilic parts of the surfactant molecule according to the following formula:

$$HLB = 20 * {M}_{h} / M$$

where Mₕ is the molecular weight of the hydrophilic portion of the molecule, and M is the molecular weight of the entire molecule, giving a result on a scale from 0 to 20. An HLB value of 0 corresponds to a completely lipophilic molecule, and a value of 20 corresponds to a completely hydrophilic molecule. Such a method for determining the HLB is described, for example, in Griffin, William C, Calculation of HLB Values of Non-Ionic Surfactants, Journal of the Society of Cosmetic Chemists, 1954, 5 (4): 249-56.

### Composition According to the Invention

According to a first aspect, the invention relates to a composition in the form of an oil-in-water microemulsion comprising at least:
a) a medium-chain fatty acid triglyceride;
b) a low HLB surfactant,
c) a high HLB surfactant;
d) a diol chosen from 1,3-propanediol and propylene glycol; and
e) water.

The medium-chain fatty acid triglycerides used in the composition according to the present invention are esters derived from the combination of glycerol and medium-chain fatty acids. They can be of natural, semi-synthetic, or synthetic origin and may include mixtures of different medium-chain fatty acid triglycerides.

In the sense of the invention “medium-chain fatty acid” means a fatty acid including 6 to 12 carbon atoms, such as caproic acid (hexanoic acid, C6), caprylic acid (octanoic acid, C8), capric acid (decanoic acid, C10), and lauric acid (dodecanoic acid, C12).

In the sense of the invention, the expression “medium-chain fatty acid triglyceride” therefore refers to a triglyceride composed of fatty acids containing 6 to 12 carbon atoms.

Preferably, the medium-chain fatty acid triglycerides are caprylic (octanoic, C8) and capric (decanoic, C10) acid triglycerides, notably called caprylic/capric triglycerides.

These triglycerides are generally obtained from vegetable oils, such as coconut oil and palm kernel oil. Caprylic/capric triglycerides may comprise from 50 to 100% by weight of caprylic acid and from 0 to 50% by weight of capric acid triglycerides.

Examples of caprylic/capric triglycerides include those available under the trade names MYRITOL (Ami Ingredients); CAPTEX (Karlshams Lipid Specialties), for example, CAPTEX 355, CAPTEX 300, CAPTEX 350, CAPTEX 850, and CAPTEX 8000; DUB MCT 5545 (Stearinerie Dubois); Crodamol™ GTCC (Croda); Labrafac CC MB (Gattefosse); SABODERM TCC MB (SABO S.p.A).

Advantageously, the composition in the form of an oil-in-water microemulsion according to the invention comprises from 3 to 15% by weight, preferably from 5 to 12% by weight, more preferably from 7 to 9% of medium-chain fatty acid triglycerides relative to the total weight of the composition.

Advantageously, the low HLB surfactant used in the composition according to the present invention comprises or is a surfactant or a mixture of surfactants having an HLB value ranging from 3 to 6, preferably from 5 to 6.

Advantageously, the low HLB surfactant is chosen from monoglycerides and diglycerides of medium-chain fatty acids, as well as mixtures thereof. The mono- and di-glycerides may each include mixtures of different mono- and di-glycerides of medium-chain fatty acids.

Preferably, the low HLB surfactant is chosen from glyceryl monocaprate, glyceryl dicaprate, glyceryl monocaprylate, glyceryl dicaprylate, glyceryl caprate, glyceryl caprylate, glyceryl monocaprylate/caprate, glyceryl caprylate/caprate, glyceryl dicaprylate/caprate, or mixtures thereof.

Examples of these monoglycerides and diglycerides of medium-chain fatty acids include those available under the trade names SYMLITE G8 (Symrise); CAPMUL (Karlsham Lipid Specialties), for example, the products CAPMUL MCM and CAPMUL C8; Cosphaderm® GMCY RSPO/MB (Cosphatec GmbH); Glyceryl Caprylate (Cosroma). Generally, these surfactants may also comprise a small amount by weight of free medium-chain fatty acids.

According to a preferred embodiment, the low HLB surfactant is glyceryl caprylate.

According to a preferred embodiment, the mass ratio of medium-chain fatty acid triglycerides/low HLB surfactant is less than 1, preferably less than 0.8, more preferably less than 0.7, and preferably between 0.40 and 0.65.

Advantageously, the composition in the form of an oil-in-water microemulsion according to the invention comprises from 5 to 25% by weight, preferably from 7 to 20% by weight, more preferably from 12 to 15% of the low HLB surfactant relative to the total weight of the composition.

Advantageously, the high HLB surfactant used in the composition according to the invention comprises or is a surfactant or a mixture of surfactants having an HLB value ranging from 13 to 20, preferably from 14 to 18.

Advantageously, the high HLB surfactant comprises one or more polyethoxylated sorbitan esters, also known as polysorbates. Among these polyethoxylated sorbitan esters, polysorbate 20: polyethoxylated sorbitan monolaurate; polysorbate 40: polyethoxylated sorbitan monopalmitate, polysorbate 60: polyethoxylated sorbitan monostearate; and polysorbate 80: polyethoxylated sorbitan monooleate can be mentioned.

According to a preferred embodiment, the low HLB surfactant is polysorbate 60 or polysorbate 80 or a mixture of both, preferably polysorbate 80.

Advantageously, the mass ratio of high HLB surfactant/(medium-chain fatty acid triglyceride+low HLB surfactant) in the composition according to the invention is greater than or equal to 0.85.

Preferably, the mass ratio of high HLB surfactant/(medium-chain fatty acid triglyceride+low HLB surfactant) ranges from 0.9 to 5, preferably from 0.9 to 2, more preferably from 0.95 to 1.5.

Advantageously, the weight amount of high HLB surfactant in the composition according to the invention is greater than that of the low HLB surfactant, which is itself greater than that of the medium-chain fatty acid triglyceride.

According to a preferred embodiment, the mass ratio of high HLB surfactant/medium-chain fatty acid triglyceride is greater than or equal to 2, preferably greater than or equal to 2.5, preferably between 2.5 and 5.

According to a particular embodiment, the mass ratio of high HLB surfactant/medium-chain fatty acid triglyceride is greater than or equal to 2 and the mass ratio of high HLB surfactant/(medium-chain fatty acid triglyceride+low HLB surfactant) in the composition according to the invention is greater than or equal to 0.85.

Advantageously, the mass ratio of high HLB surfactant to low HLB surfactant in the composition according to the invention is between 1.5 and 3, preferably between 1.5 and 2, more preferably between 1.6 and 1.8.

Advantageously, the composition in the form of an oil-in-water microemulsion according to the invention comprises from 5 to 30% by weight, preferably from 15 to 25% by weight, more preferably from 18 to 24% by weight, and more preferably from 20 to 24% by weight of the high HLB surfactant relative to the total weight of the composition.

The composition according to the invention also comprises a diol that can be chosen from 1,3-propanediol and propylene glycol (also known as propane-1,2-diol, 1,2-dihydroxypropane, or methyl glycol). Preferably, the diol is 1,3-propanediol.

According to a preferred embodiment, the mass ratio of diol/medium-chain fatty acid triglyceride is greater than or equal to 0.6, preferably greater than or equal to 0.9, more preferably greater than or equal to 1, and more preferably between 1 and 2.

Advantageously, the composition according to the invention comprises from 3% to 20% by weight, preferably from 3% to 15% by weight, more preferably from 4% to 12% by weight, more preferably from 5% to 10% by weight of diol relative to the total weight of the composition.

According to a particular embodiment, the composition according to the invention comprises from 3% to 20% by weight of diol and the mass ratio of high HLB surfactant/(medium-chain fatty acid triglyceride+low HLB surfactant) in the composition according to the invention is greater than or equal to 0.85.

The water used in the composition according to the invention can be distilled water, deionized water, or osmosis water. Water suitable for the invention can also be natural spring water or floral water.

Advantageously, the composition according to the invention comprises at least 40% by weight, preferably between 40% and 80%, more preferably between 42 and 56% by weight of water relative to the total mass of the composition.

According to a particularly preferred embodiment of the invention, the composition comprises at least:
from 7% to 9% by weight of medium-chain fatty acid triglyceride;
from 12% to 15% by weight of low HLB surfactant;
from 20% to 24% by weight of high HLB surfactant;
from 5% to 10% by weight of diol; and
from 42% to 56% by weight of water.

According to one embodiment, the composition according to the invention consists essentially of:
a medium-chain fatty acid triglyceride;
a low HLB surfactant;
a high HLB surfactant;
a diol chosen from 1,3-propanediol and propylene glycol; and
water

According to a particularly preferred embodiment of the invention, the composition consists essentially of:
from 7% to 9% by weight of medium-chain fatty acid triglyceride
from 12% to 15% by weight of low HLB surfactant;
from 20% to 24% by weight of high HLB surfactant;
from 5% to 10% by weight of diol chosen from 1,3-propanediol and propylene glycol; and
from 42% to 56% by weight of water.

The composition in the form of a microemulsion of the present invention is transparent or translucent, thus non-opaque.

According to the invention “transparent or translucent microemulsion” means a microemulsion whose matrix allows light to pass through without causing deviation by refraction or reflection, or causing only slight deviations of light rays at the interface of the two lipophilic and hydrophilic phases.

The term “transparent” refers to the physical property allowing light to pass through a material without being scattered, following Snell's law, which allows clear vision through this material. The term “translucent” includes transparency but allows light to pass with partial dispersion. This creates a blurred or veiled appearance, as the light is partially scattered when passing through the material.

The transparency or translucency of the composition according to the invention can be evaluated with the naked eye or measured using a turbidimeter.

Preferably, the diameter of the droplets of the lipophilic phase in the microemulsion of the present invention is less than 100 nm, preferably less than 80 nm, more preferably less than 50 nm, and preferably less than 40 nm.

The size of the droplets of the lipophilic phase can be determined as the average Z size or intensity-weighted average size, using techniques well known to those skilled in the art, particularly by dynamic light scattering (DLS).

The composition according to the invention exhibits excellent stability over time, particularly during transport and storage.

In the sense of the present invention, “stability” or “stable composition” means the absence of creaming (rising to the surface) or sedimentation (settling to the bottom) of the droplets of the dispersed phase in the continuous phase, the absence of coalescence (fusion of droplets), the absence of opacification of the continuous phase, and/or the absence of leakage of materials from the dispersed phase to the continuous phase, or vice versa.

In particular, the composition according to the invention has the advantage of remaining transparent or translucent at low temperature and at room temperature, without phase separation, cloudiness, or precipitation, even over very long periods.

Advantageously, the composition according to the invention can be stored and/or transported in a stable form at various temperatures, particularly at low temperature, for example from 0 to 15° C., at room temperature, for example from 15° C. to 37° C., and at high temperature, for example from 40 to 60° C.

Advantageously, the composition according to the invention can be stored and/or transported in a stable form for at least 3 months, preferably at least 6 months, more preferably 12 months, more preferably 24 months, and even better at least 36 months or better at 72 months.

According to one embodiment, the composition according to the invention can be stored and/or transported in a stable form for at least 3 months, preferably at least 6 months, more preferably 12 months, more preferably 24 months, and even better at least 36 months at a temperature ranging from 0 to 15° C.

According to another embodiment, the composition according to the invention can be stored and/or transported in a stable form for at least 3 months, preferably at least 6 months, more preferably 12 months, more preferably 24 months, and even better at least 36 months at a temperature of 15 to 37° C.

According to another embodiment, the composition according to the invention can be stored and/or transported in a stable form for at least 3 months, preferably at least 6 months, more preferably 12 months, more preferably 24 months, and even better at least 36 months at a temperature of 40 to 60° C.

Advantageously, the composition according to the invention further comprises at least one cosmetically and/or dermatologically acceptable agent.

The term “cosmetically and/or dermatologically acceptable” means a compound and/or medium that is generally safe, non-toxic, and neither biologically nor otherwise undesirable, particularly suitable for cosmetic and/or dermatological application, notably topically.

In particular, the cosmetically and/or dermatologically acceptable agent can be chosen from thickeners, preservatives, fragrances, essential oils, colorants, gelling agents, pigments, ceramics, humectants, stabilizers, emollients, and modifiers of pH, osmotic strength, and refractive index, and/or other cosmetic additives.

The composition according to the invention may also comprise at least one biological active, notably chosen from moisturizing, healing, free radical scavenging, depigmenting, melanin-regulating, UV filters, tanning accelerators, exfoliants, antioxidants, dermo-relaxing, antiperspirants, soothing, anti-aging, slimming, anti-acne, anti-seborrheic, anti-dandruff, anti-aging, anti-wrinkle, keratolytic, anti-inflammatory, refreshing, anti-hair loss, antibacterial, antifungal, antiperspirant agents, as well as their mixtures.

According to a preferred embodiment, the composition according to the invention comprises a biological active chosen from depigmenting, anti-acne, anti-aging, anti-wrinkle agents, and their mixtures.

According to one embodiment, the composition according to the invention further comprises a depigmenting active chosen from hydroquinone derivatives, such as arbutin, deoxyarbutin, and alpha-arbutin; ascorbic acid, tranexamic acid and its derivatives, glycolic acid, kojic acid, azelaic acid and its derivatives, retinoic acid, niacinamide, glutathione, polyphenols, such as resveratrol and isoflavones like glabridin (licorice root extract), resorcinol derivatives, such as phenylethyl resorcinol, as well as mixtures of these actives.

Preferably, the depigmenting active is chosen from cetyl tranexamate mesylate, glutathione, phenylethyl resorcinol, and their mixtures.

According to another preferred embodiment, the composition according to the invention further comprises an anti-aging or anti-wrinkle active chosen from vitamin A and its derivatives (retinol, tretinoin (retinoic acid)), matrixyl (palmitoyl pentapeptide-4), hyaluronic acid, vitamin C (ascorbic acid), vitamin E (tocopherol), AHAs (alpha-hydroxy acids), such as glycolic acid and BHAs (beta-hydroxy acids), coenzyme Q10, vitamin B3 (niacinamide), green tea extract, alpha-lipoic acid, as well as their derivatives and mixtures. Preferably, the skin anti-aging active is chosen from retinoic acid ester, acetyl hexapeptide-8, hydroxyprolisilane, and their mixtures.

According to another preferred embodiment, the composition according to the invention further comprises an anti-acne active chosen from salicylic acid, shikimic acid, benzoyl peroxide, retinoids, niacinamide, azelaic acid and its derivatives, glycolic acid, sulfur, green tea extract, clindamycin, rose water, zinc, PHAs (poly-hydroxy acids) as well as their derivatives and mixtures. Preferably, the anti-acne active is chosen from shikimic acid, succinic acid, capryloyl salicylic acid, and their mixtures.

Preferably, the composition according to the invention comprises from 0.01 to 15% by weight, preferably from 0.5 to 10% by weight, more preferably from 1 to 5% by weight of one or more biological actives as described above.

The cosmetically and/or dermatologically acceptable agents or biological actives can be added to the composition in free or encapsulated form.

Of course, those skilled in the art will know how to choose the possible additional compound(s) and/or their quantity so that the advantageous properties and stability of the composition according to the invention are not or are substantially not altered.

The composition according to the invention may also include one or more pharmaceutical active ingredients.

The composition in the form of a microemulsion according to the invention can be prepared by mixing the different compounds as defined above by techniques known to those skilled in the art. The order of addition of the compounds, the speed, or the type of mixing are not critical for the preparation of the composition according to the invention.

Preferably, the oil-in-water microemulsion according to the present invention forms spontaneously when the ingredients as defined above are brought together. It is known that the microemulsion is formed when the mixture becomes transparent or translucent. Preferably, the preparation process of the composition according to the invention does not comprise a heating step.

The composition can be prepared, for example, by gradually adding the high HLB surfactant to a mixture comprising the medium-chain fatty acid triglyceride, 1,3-propanediol, the low HLB surfactant, and optionally additives or biological actives, while stirring the reaction medium.

### Uses of the Composition According to the Invention

The invention also relates to the use of a composition in the form of an oil-in-water microemulsion as described in detail above as a care and/or hygiene and/or makeup product for keratinous materials, notably the skin and hair.

In the sense of the invention “keratinous materials” means the skin, notably of the body or face, hair, nails, scalp, and hair.

The composition according to the invention can thus be used as a base for products in the form of a liquid lotion, serum, mist, or micellar water. The composition according to the invention can also be used to impregnate makeup removal wipes.

The composition according to the invention can be used in all external cutaneous (topical) and/or internal (oral, injectable) applications. Preferably, the composition is used for external topical use.

The invention also relates to the non-therapeutic cosmetic use of a composition as described above to improve skin firmness and/or elasticity and/or improve skin microrelief and/or improve skin radiance.

The invention also relates to the non-therapeutic cosmetic use of a composition as described above to prevent and/or treat signs of skin aging, preferably to reduce and/or eliminate and/or attenuate wrinkles and fine lines.

The invention also relates to the non-therapeutic cosmetic use of a composition as described above to prevent and/or reduce and/or eliminate acne, particularly the bacterium Cutibacterium acnes, and/or to promote the elimination of cosmetic effects of acne, particularly acne spots.

The invention also relates to the non-therapeutic cosmetic use of a composition as described above to prevent and/or reduce and/or attenuate and/or eliminate pigmentation spots, and/or reduce their size, and/or unify and/or illuminate and/or brighten the complexion.

The invention also relates to a non-therapeutic cosmetic treatment method consisting of applying the composition according to the invention to keratinous materials, notably the skin.

### Examples

The invention is illustrated by the following examples given as non-limiting.

In the examples, parts and percentages are expressed by weight unless otherwise indicated.

### 1—Materials and Methods

### Preparation of Microemulsions:

The compositions according to the invention were prepared by making a first mixture of water and 1,3-propanediol (M1) and a second mixture comprising glyceryl caprylate, caprylic/capric triglyceride, and polysorbate 80 (M2). The two solutions, M1 and M2, were stirred until homogenization, then the M2 mixture was slowly added to the M1 mixture while continuing to stir with a magnetic stirrer. The list of ingredients used is presented in Table 1.

TABLE 1
Ingredients Used and Their INCI Names

| | | Commercial Name and |
|---|---|---|
| Ingredient | INCI Name | Manufacturer |
| Osmosis Water | WATER | Dermosciences |
| High HLB Surfactant | POLYSORBATE 80 | EUMULGIN SMO 20, |
| | | BASF |
| Medium-Chain Fatty | CAPRYLIC/CAPRIC | MYRITOL 318, Ami |
| Acid Triglyceride | TRIGLYCERIDE | Ingredients |
| Diol | 1,3-PROPANEDIOL | TILAMAR PDO WITH |
| | | NOOVISTA, Ies |
| | | Ingredients |
| Low HLB Surfactant | GLYCERYL CAPRYLATE | SYMLITE G8, Symrise |

### Stability Measurement Protocol for Microemulsions:

Stability tests were conducted over a period of 6 months under accelerated conditions at 5° C.±3° C., 25° C.±5° C., and 45° C.±2° C. The objective of this accelerated stability test is to determine the degradation time of the microemulsion. About 15 ml of each prepared composition is transferred into 3 non-amber glass jars of 30 ml. Each jar was stored both in a room where the temperature is set at 25° C.±5° C. and in ovens at 5° C.±3° C. (“Aqualytic Refrigerator TC 135 S” oven) and at 45° C.±2° C. (“Memmert Heating Oven UF55” oven).

The appearance, color, and odor are evaluated at the following deadlines: 7 days, 14 days, 1 month, 1 and a half months, 3 months, and 6 months. At each deadline, the compositions were examined with the naked eye upon exiting the oven and once returned to room temperature (after a few hours of rest).

If, for example, the composition stored at 45° C. remains stable for three months, it can be granted a stability duration of up to 36 months. If the composition stored at 45° C. remains stable for six months, it can be granted a stability duration of up to 6 years.

### 2—Results

The different compositions tested as well as the stability results are presented in Table 2. Compositions E15, E31, E32, and E33 are according to the invention. Composition E13 is comparative.

Emulsions are considered stable in storage if they do not show signs of instability (change in color, odor, and/or transparent appearance) up to 3 months of storage at 45° C. The results show that composition E13, without diol, loses its stability after 2 months at 45° C. In contrast, compositions E15, E31, E32, and E33, according to the invention, remain stable for 3 months at 45° C., which corresponds to a stability of 3 years at room temperature.

Other compositions E17, E18, E19, E20, and E30 were also evaluated by varying the mass ratio between the high HLB surfactant and the mixture (medium-chain fatty acid triglyceride+low HLB surfactant). These compositions with a ratio below 0.85 show earlier signs of instability, even at room temperature.

TABLE 2
Stability of Tested Compositions

| | E13 | E15 | E31 | E32 | E33 |
|---|---|---|---|---|---|
| Caprylic/Capric | 7.5 | 7.5 | 7.5 | 7.5 | 8.5 |
| Triglyceride (a) (%) | | | | | |
| Glyceryl Caprylate | 12.5 | 12.5 | 12.5 | 12.5 | 14 |
| (b) (%) | | | | | |
| Polysorbate 80 (c) | 20 | 20 | 20 | 20 | 22.5 |
| (%) | | | | | |
| 1,3-propanediol (d) | 0 | 5 | 7.5 | 10 | 10 |
| (%) | | | | | |
| Water (e) (%) | 60 | 55 | 52.5 | 50 | 45 |
| Ratio (c)/((a) + (b)) | 1 | 1 | 1 | 1 | 1 |
| Stability | 2 months | 3 months | 6 months | 6 months | 6 months |
| | at 45° C. | at 45° C. | at 45° C. | at 45° C. | at 45° C. |

| | E17 | E18 | E19 | E20 | E30 |
|---|---|---|---|---|---|
| Caprylic/Capric | 7.5 | 7.5 | 8.5 | 8.5 | 12.5 |
| Triglyceride (a) (%) | | | | | |
| Glyceryl Caprylate | 12.5 | 12.5 | 14 | 14 | 12.5 |
| (b) (%) | | | | | |
| Polysorbate 80 (c) | 12.5 | 15 | 15 | 12.5 | 20 |
| (%) | | | | | |
| 1,3-propanediol (d) | 2.5 | 5 | 5 | 5 | 5 |
| (%) | | | | | |
| Water (e) (%) | 65 | 60 | 57.5 | 60 | 50 |
| Ratio (c)/((a) + (b)) | 0.63 | 0.75 | 0.67 | 0.56 | 0.80 |
| Stability | 4° C.: | 2 months | J14 at | 25° C.: | 4° C.: |
| | cloudy | at 45° C.: | 45° C.: | cloudy | cloudy |
| | | cloudy | cloudy | | |

1. A composition in the form of an oil-in-water microemulsion comprising at least:
a) a medium-chain fatty acid triglyceride having 6 to 12 carbon atoms;
b) a low HLB surfactant having an HLB ranging from 3 to 6;
c) a high HLB surfactant having an HLB ranging from 13 to 20;
d) a diol chosen from 1,3-propanediol and propylene glycol;
e) water,
wherein the mass ratio of the high HLB surfactant to the mixture (medium-chain fatty acid triglyceride having 6 to 12 carbon atoms+low HLB surfactant) is greater than or equal to 0.85.

2. The composition according to claim 1, wherein the low HLB surfactant has an HLB ranging from 5 to 6 and/or the high HLB surfactant has an HLB ranging from 14 to 18.

3. The composition according to claim 1, wherein the mass ratio of the high HLB surfactant to the mixture (medium-chain fatty acid triglyceride+low HLB surfactant) ranges from 0.90 to 5, preferably from 0.90 to 2.

4. The composition according to claim 1, wherein the mass ratio of medium-chain fatty acid triglycerides/low HLB surfactant is less than 1, preferably less than 0.8, more preferably less than 0.7, and better between 0.40 and 0.65.

5. The composition according to claim 1, wherein the mass ratio of high HLB surfactant/low HLB surfactant is between 1.5 and 3, preferably between 1.5 and 2, more preferably between 1.6 and 1.8.

6. The composition according to claim 1, wherein the mass ratio of diol/medium-chain fatty acid triglyceride is greater than or equal to 0.6, preferably greater than or equal to 0.9, more preferably greater than or equal to 1, and more preferably between 1 and 2.

7. The composition according to claim 1, comprising from 3% to 20% by weight, preferably from 3% to 15% by weight, more preferably from 4% to 12% by weight, more preferably from 5% to 10% by weight of diol relative to the total weight of the composition.

8. The composition according to claim 1, wherein the diol is 1,3-propanediol.

9. The composition according to claim 1, wherein the medium-chain fatty acid triglyceride having 6 to 12 carbon atoms is caprylic/capric triglyceride.

10. The composition according to claim 1, wherein the low HLB surfactant comprises a monoglyceride and/or a diglyceride of medium-chain fatty acid chosen from glyceryl monocaprate, glyceryl dicaprate, glyceryl monocaprylate, glyceryl dicaprylate, glyceryl caprate, glyceryl caprylate, glyceryl monocaprylate/caprate, glyceryl caprylate/caprate, glyceryl dicaprylate/caprate, preferably glyceryl caprylate.

11. The composition according to claim 1, wherein the high HLB surfactant comprises an ethoxylated sorbitan ester.

12. The composition according to claim 11 where the high HLB surfactant is polysorbate 80.

13. The composition according to claim 1, comprising, relative to the total weight of the composition:
a) from 7% to 9% by weight of triglyceride;
b) from 12% to 15% by weight of low HLB surfactant;
c) from 20% to 24% by weight of high HLB surfactant;
d) from 5% to 10% by weight of diol; and
e) from 42% to 56% by weight of water.

14. The composition according to claim 1, further comprising at least one cosmetically and/or dermatologically acceptable agent and/or at least one biological active.

15. A use of a composition according to claim 1 as a makeup, hygiene, cleaning, and/or care product for keratinous materials, preferably the skin.
